(19) United States
(12) Patent Application Publication
(10) Pub. No.: US 2007/0288039 A1
(43) Pub. Date: Dec. 13, 2007

(54) Clip applying apparatus with curved jaws, and clip

(75) Inventors: Ernest Aranyi, Easton, CT (US); Kenneth H. Whitfield, New Haven, CT (US)

Correspondence Address: COVIDIEN, 60 MIDDLETOWN AVENUE, NORTH HAVEN, CT, 06473, US

(73) Assignee: Tyco Healthcare Group LP

(21) Appl. No.: 11/728,699

(22) Filed: Mar. 27, 2007

Related U.S. Application Data
(62) Division of application No. 10/798,028, filed on Mar. 11, 2004, now abandoned.
(60) Provisional application No. 60/453,586, filed on Mar. 11, 2003.

(51) Int. Cl. A61B 17/122

(52) U.S. Cl. 606/144

(57) ABSTRACT

A clip applying apparatus is provided which includes a jaw mechanism supported within and extending from the distal end of a body portion of the apparatus. The jaw mechanism includes a proximal body portion, first and second shank members and first and second jaw members. The first and second shank members extend distally from the proximal body portion of the jaw mechanism. The first and second jaw members are supported on the distal end of the first and second shank members and are configured to receive a ligation clip therebetween. In a particularly useful embodiment, each jaw is curved upwardly towards its distal end along the longitudinal axis of the body portion and has a radius of curvature “r” of between about 0.5 inch and about 0.9 inch. In a particularly useful embodiment, r is about 0.7 inch. A method for accessing and ligating tissue is also disclosed wherein the clip applying apparatus described above is positioned adjacent tissue to be ligated and the jaws are moved between the tissue to be ligated and surrounding tissue to manipulate and/or reposition the tissue. The jaw mechanism can be rotated to position the tissue to be ligated between the first and second jaws to ligate and/or crimp a ligation clip about the tissue to be ligated.

This application is a divisional of U.S. patent application Ser. No. 10/798,028 filed on Mar. 11, 2004, which claims priority to U.S. Provisional Application Ser. No. 60/453,586 filed Mar. 11, 2003. The entire contents of each of these applications is incorporated herein by reference.

### BACKGROUND

1. Technical Field

The present disclosure relates to clip applying apparatus and ligation clips. More particularly, the present disclosure relates to a clip applying apparatus for applying surgical clips to body tissue having a jaw mechanism configured to more easily access tissue to be ligated.

2. Background to Related Art

Surgical procedures frequently require ligation of blood vessels, severed tissues and/or other organs to control and stop bleeding. Clip applying apparatus for quickly applying a surgical clip about tissue are well known. During endoscopic surgical procedures, the body portion of a clip applying apparatus is introduced into a body cavity through a small opening in body tissue, generally, via a trocar cannula assembly. Thereafter, a surgeon manipulates a handle portion of the apparatus which is positioned externally of the body cavity to access tissue.

Typically, clip applying apparatus include a jaw mechanism, e.g., a pair of jaws, supported at the distal end of an elongated body portion of the clip applying apparatus. The jaw mechanism is configured to receive, retain, crimp and release a ligation clip from the apparatus. During use, the jaw mechanism is positioned about tissue to be ligated and closed to deform the ligation clip about tissue.

One problem associated with current clip applying apparatus and especially endoscopic clip applying apparatus is difficulty in accessing tissue, e.g., a vessel, and properly positioning the jaw mechanism about the tissue to be ligated. Accordingly, a continuing need exists for a clip applying apparatus having jaw structure configured to more easily access and position the jaw mechanism about tissue to be ligated.

### SUMMARY

A clip applying apparatus is provided which includes a handle assembly, a body portion and a jaw mechanism. The handle assembly includes a stationary handle and an actuation member, e.g., a pivotable trigger. The body portion defines a longitudinal axis and extends distally from the handle assembly. The jaw mechanism is supported within and extends from the distal end of the body portion and includes a proximal body portion, first and second shank members and first and second jaw members. The jaw mechanism may be of monolithic construction or constructed from separate parts. The first and second shank members extend distally from the proximal body portion of the jaw mechanism. The first and second jaw members are supported on the distal end of the first and second shank members. Each shank member defines a cam surface. A closure member is movable in response to movement of the actuation member through an actuation stroke into engagement with the cam surfaces of the first and second shank members to move the first and second jaws from an open spaced position to a closed approximated position.

The first and second jaws are configured to receive a ligation clip therebetween. In a particularly useful embodiment, each jaw is curved upwardly towards its distal end along the longitudinal axis of the body portion. Preferably, each jaw has a radius of curvature “r” of between about 0.5 inch and about 0.9 inch. In a particularly useful embodiment, r is about 0.7 inch.

In another particularly useful embodiment, the body portion is operably connected to a rotatable knob which is rotatably supported on the handle assembly. The rotatable knob is rotatable to rotate the body portion and the jaw mechanism about the longitudinal axis of the body portion.

A method for accessing and ligating tissue is also disclosed wherein the clip applying apparatus described above is positioned adjacent tissue to be ligated. This can be accomplished by inserting the jaw mechanism directly through an opening formed in body tissue or through a cannula assembly. Next, the jaws are moved between the body tissue to be ligated and surrounding tissue and the apparatus is manipulated to reposition the tissue to be ligated. Finally, the jaw mechanism is rotated to position the tissue to be ligated between the first and second jaws and the jaws are moved from the open position to the closed position to crimp a ligation clip about the tissue to be ligated.

### BRIEF DESCRIPTION OF THE DRAWINGS

Various preferred embodiments of the presently disclosed clip applying apparatus are described herein with reference to the drawings, wherein:

FIG. 1 is a side perspective view of one embodiment of the presently disclosed clip applying apparatus showing a preferred jaw structure;

FIG. 2 is an enlarged view of the indicated area of detail of the jaw structure shown in FIG. 1;

FIG. 3 is a top, side perspective view with portions broken away of the jaw structure and a surgical clip of the clip applying apparatus shown in FIG. 1;

FIG. 4 is a side view of the jaw structure and surgical clip of the clip applying apparatus shown in FIG. 3;

FIG. 5 is a top side perspective view of a surgical clip of the clip applying apparatus shown in FIG. 1;

FIG. 6 is a perspective, partial cutaway view of the elongated body and jaw structure of the clip applying apparatus shown in FIG. 1 extending through a cannula assembly;

FIG. 7 is a cross-sectional view of a vessel to be ligated embedded in body tissue;

FIG. 8 is a cross-sectional view of the vessel shown in FIG. 7 being accessed through a cannula by the distal end of the clip applying apparatus shown in FIG. 1;

FIG. 9 is a cross-sectional view of the vessel shown in FIG. 8 being manipulated through a cannula by the distal end of the clip applying apparatus;

FIG. 10 is a cross-sectional view of the clip applying apparatus and vessel shown in FIG. 9 with the central body portion and jaw mechanism of the apparatus rotated;

FIG. 11 is a cross-sectional view of the clip applying apparatus and vessel shown in FIG. 10 with the jaw structure and ligation clip positioned about the vessel;

FIG. 12 is a top, cross-sectional view of the distal end of the central body portion and jaw structure of the clip applying apparatus shown in FIG. 1 with a clip positioned within the jaws and the jaws in an open position;

FIG. 13 is a top, cross-sectional view of the distal end of the central body portion and jaw structure of the clip applying apparatus shown in FIG. 12 with a clip positioned within the jaws and the jaws in a closed position;

FIG. 14 is a side view with portions broken away of a deformed ligation clip crimped about a vessel; and

FIG. 15 is a perspective view of the ligation clip and vessel shown in FIG. 14.

### DETAILED DESCRIPTION OF PREFERRED EMBODIMENTS

Preferred embodiments of the presently disclosed clip applying apparatus and ligation clip will now be described in detail with reference to the drawings, in which like reference numerals designate identical or corresponding elements in each of the several views.

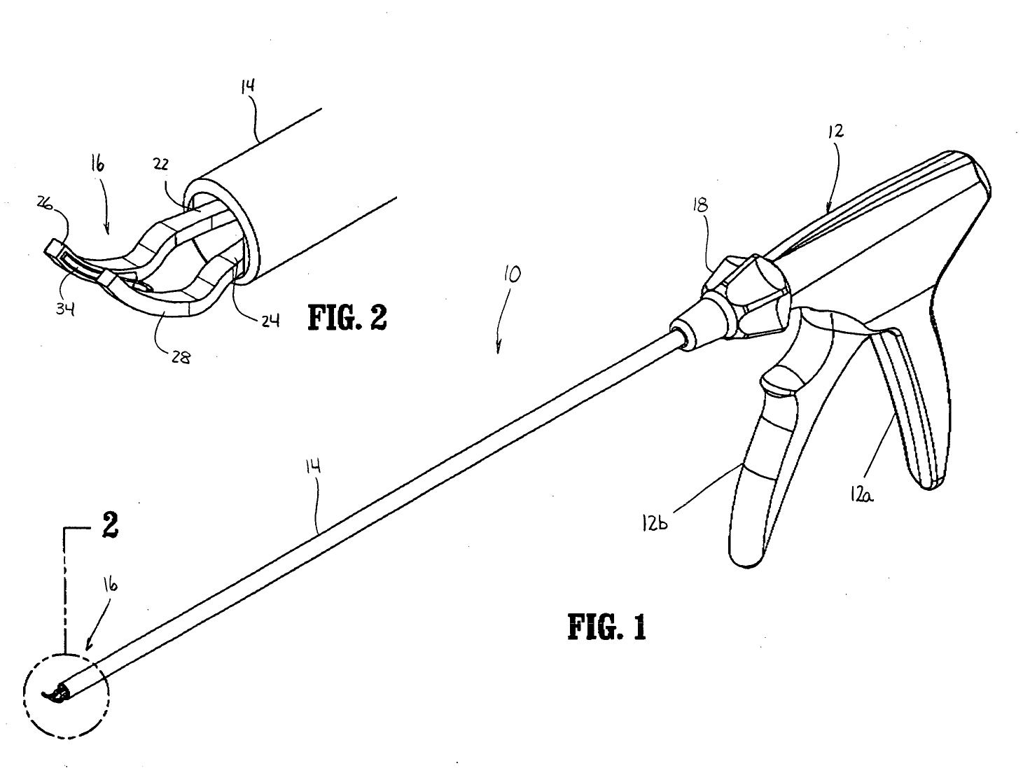

FIG. 1 illustrates one preferred embodiment of the presently disclosed clip applying apparatus 10. Briefly, clip applying apparatus 10 includes a handle assembly 12 including a stationary handle 12a and a pivotable trigger or actuation member 12b, a central body portion 14 and jaw mechanism 16. Although handle assembly 12 is illustrated having a pistol grip configuration other known handle configurations are envisioned, e.g., in-line handle, scissors handle, tweezers handle, etc. A rotatable knob 18 is rotatably supported on a distal end of handle assembly 12. Rotatable knob 18 supports the proximal end of central body portion 14 in a known manner such that rotatable knob 18, central body portion 14 and jaw mechanism 16 are rotatable in relation to handle assembly 12 about the longitudinal axis of central body portion 14. It is envisioned that the rotatable knob 18 may rotate the jaw mechanism 16 while the central body portion 14 remains stationary. Although specific details of the handle assembly are not disclosed herein, the handle assembly 12 may include any handle mechanism known in the art to effect actuation of a jaw mechanism as will be described in detail below. Examples of such known handle mechanisms are disclosed in U.S. Pat. Nos. 5,938,667, 5,868,761, 5,868,759, 5,725,538, 5,720,756, 5,700,270, 5,695,502, 5,645,553, 5,626,585, 5,591,178, 5,514,149, 5,462,558, 5,300,081, 5,197,970 and 4,509,518 which are all incorporated herein in their entirety by reference.

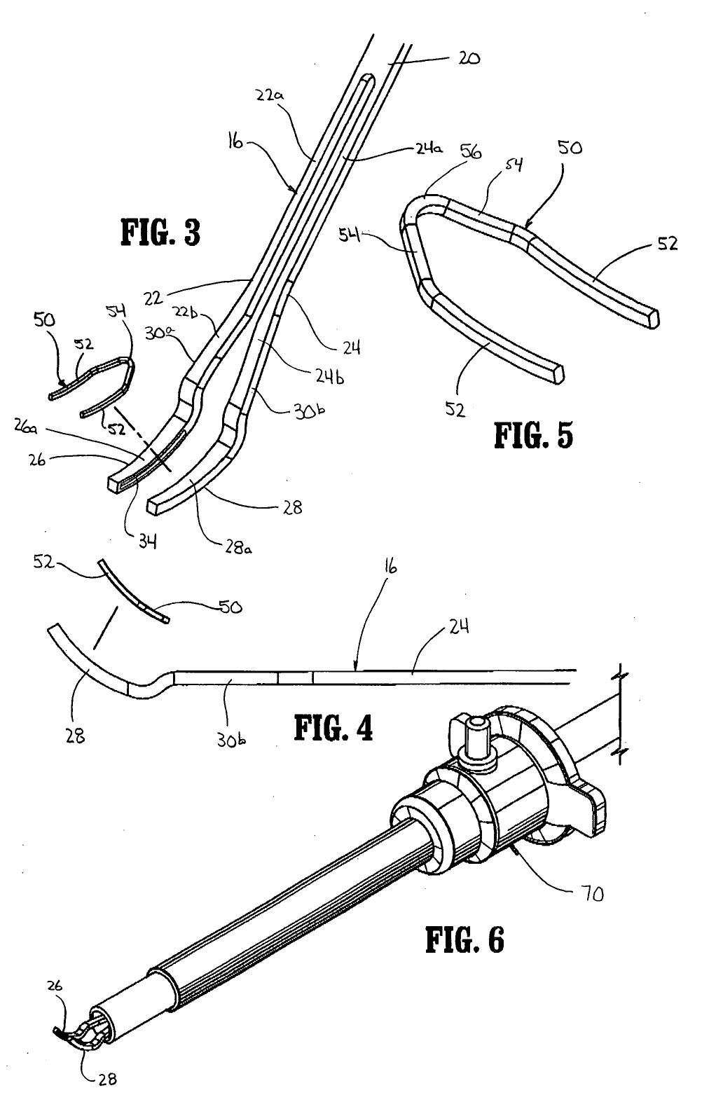

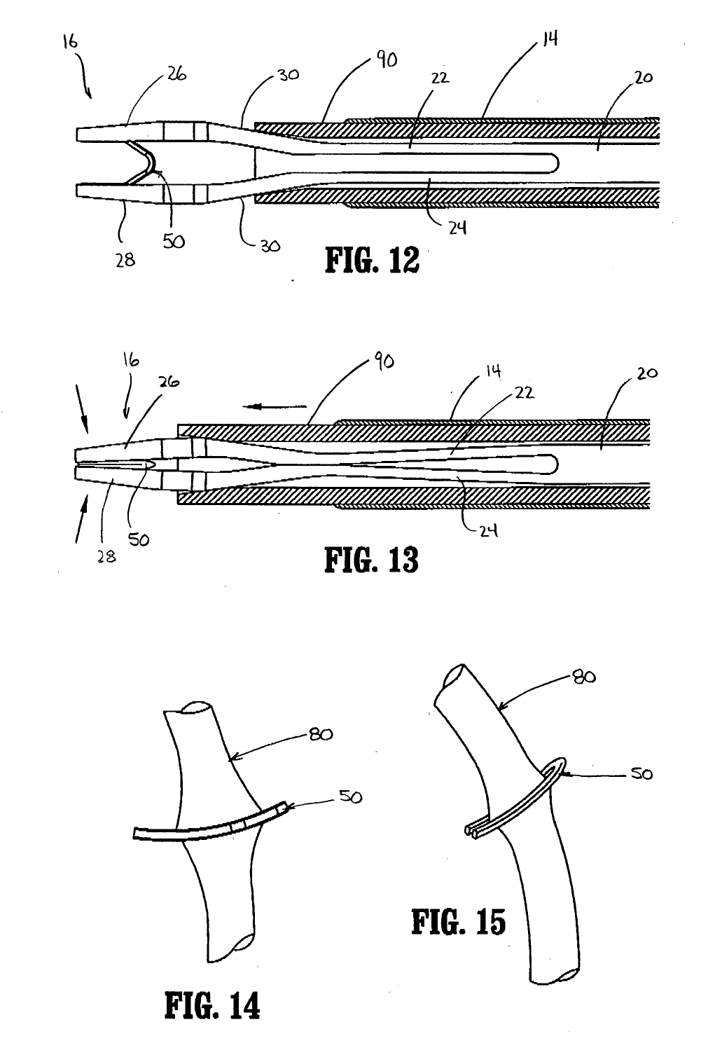

Referring to FIGS. 2, 3 and 4, jaw mechanism 16 has a longitudinal axis and includes a jaw body 20, a pair of spaced shank members 22 and 24 and jaw structure in the form of first and second jaws 26 and 28, respectively. Each shank member 22 and 24 has a proximal end which extends from jaw body 20 distally to a respective first or second jaw member. Preferably, jaw mechanism 16 is monolithically formed from a suitable surgically approved material such as spring steel. Alternately, jaw mechanism 16 may be constructed of multiple parts which are fastened together using known fastening techniques. Each shank portion 22 and 24 includes a generally parallel portion 22a and 24a and a diverging portion 22b and 24b. Diverging portions 22b and 24b include outer walls which define cam surfaces 30a and 30b, respectively. A closure member 90 is movable into engagement with cam surfaces 30a and 30b in response to movement of actuation member 12b through an actuation stroke to move shank members 22 and 24 toward each other and move first and second jaws 26 and 28 from an open position (FIG. 12) to a closed position (FIG. 13). Closure member 90 is movable within body portion 14 and is operably connected to actuation member 12b. The linkage connecting closure member 90 to actuation member 12b can be any of a variety of linkages known to those of ordinary skill in the art. It is understood that jaws 26 and 28 can be partly closed at intermediate positions between those shown in FIGS. 12 and 13.

Each first and second jaw 26 and 28 includes a body 26a and 28a, respectively, having a predefined radius of curvature “r” (FIG. 4) along the longitudinal axis of jaw mechanism 16, such that the distal end of each of jaws 26 and 28 extends upwardly from the longitudinal axis of jaw mechanism 16. In a particularly useful embodiment, r is from about 0.5 inch (about 12.7 mm) to about 0.9 inch (about 22.7 mm). Most preferably r is about 0.7 inch (about 18 mm). Each of jaws 26 and 28 includes a curved channel 34 formed along an inner sidewall thereof. Each curved channel 34 of each jaw 26 and 28 is dimensioned to slidably receive a corresponding leg 52 of a surgical clip, e.g., ligation clip 50, such that the clip 50 is supported between first and second jaws 26 and 28 when the jaws are in the open position. The overall curvature of each curved jaw 26 and 28 and/or of channels 34 need not be formed by a single radius, i.e., jaws having multiple radii are envisioned.

One preferred embodiment of clip 50 is shown in FIG. 5. Clip 50 includes a pair of legs 52 and a backspan 54. Backspan 54 is preferably triangularly shaped with a curved or rounded apex 56. Alternately, backspan 54 may have a variety of configurations including linear, curved, etc. Each leg 52 defines a longitudinal axis. The entire clip from apex 56 to the tip of each leg has a radius of curvature along its longitudinal axis that preferably is substantially the same as radius of curvature “r” of jaws 26 and 28 and/or channels 34. Thus, clip 50 can be slidably received within the jaw channels 34 of jaws 26 and 28. Alternately, clips 50 may be formed without a radius of curvature and may be provided with or deformed to have a radius of curvature as the clips 50 are supplied or fed into jaws 26 and 28 through channels 34. As can be appreciated, this would require a semi-resilient or deformable clip 50.

Clip applying apparatus 10 may be formed as a single clip applicator in which a clip 50 is manually placed between jaws 26 and 28 prior to each use of the clip applicator. It is also envisioned that clip applying apparatus 10 can be formed as a multiple clip applicator which includes a stack or series of clips 50 positioned within central body portion 14 of clip applying apparatus 10. Such a device may include clip feeding and advancing mechanisms similar to those disclosed in the U.S. patents disclosed above which have been incorporated herein by reference.

Referring to FIG. 6, clip applying apparatus 10 may be used in conjunction with a trocar cannula assembly, e.g., 70, during performance of an endoscopic surgical procedure.

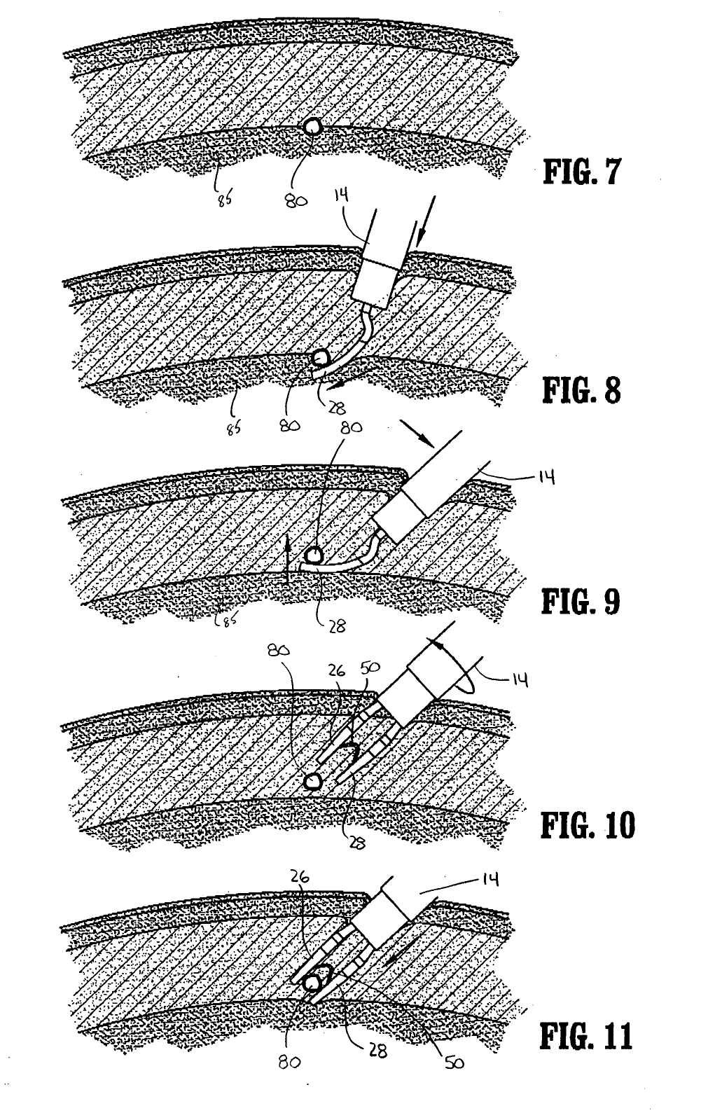

Referring to FIGS. 7-11, curved jaw mechanism 16 including curved jaws 26 and 28 provides the advantage of simplifying access to tissue, e.g., a vessel 80. For example, when a vessel 80 to be ligated is embedded in surrounding tissue 85 or at a location which is difficult to reach, the tips of jaws 26 and 28 can be slid behind or under the vessel 80 (FIG. 8) and manipulated to separate vessel 80 from surrounding tissue 85 and position vessel 80 at a more accessible location (FIG. 8). Thereafter, the position of central body portion 14 and jaw mechanism 16 of clip applying apparatus 10 can be adjusted e.g., rotated 90° (FIG. 10), to position clip 50 about vessel 80.

Referring to FIG. 12, prior to use, ligation clip 50 is positioned between jaws 26 and 28 such that legs 52 of clip 50 are supported in channels 34 of jaws 26 and 28. When pivotable trigger 12b is actuated (FIG. 1), i.e., compressed towards stationary handle 12a, closure member 90 is advanced distally in relation to jaws 26 and 28 to engage cam surfaces 30a and 30b and urge jaws 26 and 28 from the open position (FIG. 12) to the closed position (FIG. 13) to crimp and close clip 50. As illustrated in FIGS. 14 and 15, after ligation clip 50 has been fully deformed about the vessel 80 to be ligated, pivotable trigger 12b can be released to disengage clip 50 from jaws 26 and 28.

Apparatus 10 may be constructed from any suitable medical grade material including plastics and metals suitable for use in medical instrumentation. For example, the jaws 26 and 28 and other high force bearing components of clip applying apparatus 10 may be constructed from surgical grade metals, whereas other minimal force bearing components of clip applying apparatus 10 may be formed from surgically approved filled or unfilled plastics.

The benefits of the presently disclosed curved jaws of the clip applying apparatus include the improved ability to separate tissue from surrounding tissue and manipulate tissue at the surgical site. Visibility is also improved because the curved jaws 26 and 28 are less obstructed by the distal components of the apparatus.

The clip applying apparatus 10 may be sold as a kit wherein differently shaped jaw mechanisms may be selectively attached to clip applying apparatus 10 to achieve a particular purpose or to achieve a desired result. In such an apparatus, the jaws are necessarily removable from the remaining portion of the jaw mechanism and/or the jaw mechanism is removable from the apparatus. For example, a kit may include several jaw mechanisms 16, each having a different radius of curvature, to provide the surgeon greater flexibility and greater degrees of visibility when utilizing the clip applying apparatus. Further, as mentioned above, a semi-resilient, resilient or deformable clip 50 may be utilized with clip applying apparatus 10 to enable the same clip 50 (with or without pre-curved legs) to be utilized in instruments having jaws with different radius of curvatures. During positioning of the clips into the jaws, the clips would take on the curvature of the jaws and/or jaw channels in accordance with the designs employed. Alternatively, separate pre-curved clips which correspond to jaws having a particular radius of curvature may be provided. These clips may be color-coded to facilitate selection of the proper clip for a particular instrument.

Although the illustrative embodiments of the present disclosure have been described herein with reference to the accompanying drawings, it is to be understood that this disclosure in not limited to those precise embodiments and that various other changes and modifications may be affected therein by one skilled in the art without departing from the scope or spirit of the disclosure. All such changes and modifications are intended to be included within the scope of any appended claims.

What is claimed is:

1. A method of accessing and ligating tissue, the method comprising the steps of:
a) providing a clip applying apparatus having a handle assembly, a body portion defining a longitudinal axis and extending distally from the handle assembly and a jaw mechanism including first and second jaws movable in relation to each other between open and closed positions, the first and second jaws being curved upwardly towards the distal end of the jaws along the longitudinal axis of the body portion, each curved jaw having a radius of curvature “r”;
b) positioning the jaws between tissue to be ligated and surrounding tissue;
c) manipulating the jaws to reposition the tissue to be ligated;
d) rotating the jaw mechanism to position the tissue to be ligated between the first and second jaws; and
e) moving the first and second jaws from the open position to the closed position to ligate the tissue to be ligated.

2. A method according to claim 1, further including the step of:
a) inserting the body portion and jaw mechanism of the clip applying apparatus through a cannula assembly to access the tissue to be ligated.

3. A method according to claim 1, wherein r is between about 0.5 inch and 0.9 inch.

4. A method according to claim 2, wherein r is about 0.7 inch.

5. A method according to claim 1, further including the step of:
f) placing a clip between the jaws prior to moving the jaws from the open position to the closed position and thereby crimping a clip about the tissue to be ligated when the jaws are moved from the open position to the closed position.

6. A surgical clip comprising:
a pair of legs and a backspan interconnecting the pair of legs, the clip defining a longitudinal axis and having a radius of curvature along its longitudinal axis of between about 0.5 inch and about 0.9 inch.

7. A surgical clip according to claim 6, wherein the radius of curvature is about 0.7 inch.

8. A kit comprising:
a clip applying apparatus including a handle assembly and a body portion defining a longitudinal axis, the kit further including a plurality of jaw mechanisms, each of the plurality of jaw mechanisms including first and second jaws having a predefined radius of curvature, wherein the radius of curvature of the first and second jaws of each of the plurality of jaw mechanisms is different from the radius of curvature of each of the first and second jaws of each of the other of the plurality of jaw mechanisms.
